(19) United States
(12) Patent Application Publication
(10) Pub. No.: US 2018/0030123 A1
(43) Pub. Date: Feb. 1, 2018

(54) METHOD FOR TREATING A TUMOR WITH BIFUNCTIONAL AGENTS THAT BIND TO TUMOR CARBOHYDRATE ANTIGENS

(71) Applicant: Lan Bo Chen, Lexington, MA (US)

(72) Inventor: Lan Bo Chen, Lexington, MA (US)

(21) Appl. No.: 15/664,047

(22) Filed: Jul. 31, 2017

Related U.S. Application Data
(60) Provisional application No. 62/368,550, filed on Jul. 29, 2016.

(51) Int. Cl. C07K 16/18; C07K 14/54; C07K 14/55; C07K 16/28; C07K 16/30

(52) U.S. Cl. CPC C07K 16/18; C07K 16/2809; C07K 16/283; C07K 16/30; C07K 14/55; C07K 14/5434; C07K 2317/31; C07K 2317/52; C07K 2317/622; C07K 2319/00

(57) ABSTRACT

A method for treating a tumor with at least two bifunctional agents. The bifunctional agents each include a binding domain that specifically binds to stage-specific embryonic antigen 4 (SSEA4) or an SSEA4 analog, and an effector domain selected from a cytokine, a cytotoxic agent, an immunoglobulin Fc domain, anti-CD3, and anti-CD16.

### CROSS-REFERENCE TO RELATED APPLICATIONS

The present application claims the priority of Provisional Application No. 62/368,550, filed on Jul. 29, 2016. The content of this prior application is hereby incorporated by reference in its entirety.

### BACKGROUND

Chemotherapy and radiotherapy are still considered to be first-line treatments for many tumors. However, the effectiveness of these two treatment modalities is often limited by the development of resistant tumor cells and by side effects that can necessitate a halt to treatment.

Targeted therapies hold the promise of better efficacy with fewer side effects. For example, recently developed therapeutic antibodies that bind specifically to antigens on tumor cells lead to immune system-mediated death of the tumor cells via antibody-dependent cell-mediated cytotoxicity or via complement-dependent cytotoxicity.

In another approach, tumor treatment modalities have been developed that combine the advantages of specific targeting of tumor cells with immune system stimulation.

An additional approach relies on the specificity of antibodies that bind to tumor cells to deliver a cytotoxic agent to the tumor cells and not surrounding normal cells.

There is a need for tumor treatment methods that combine the advantages of multiple therapeutic approaches.

### SUMMARY

To meet this need, provided is a method for treating a tumor by administering to a subject having a tumor at least two different bifunctional agents. Each of the bifunctional agents contains a binding domain linked to an effector molecule. The binding domain specifically binds to stage-specific embryonic antigen 4 (SSEA4) or an SSEA4 analog. The effector molecule can be a cytotoxic agent, a cytokine, an immunoglobulin Fc domain, anti-CD3, and anti-CD16. Cells in the tumor express SSEA4 or the SSEA4 analog.

The tumor can be, but is not limited to a tumor of the breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow.

The details of one or more embodiments of the invention are set forth in the description below. Other features, objects, and advantages of the invention will be apparent from the description and from the claims.

Importantly, all references cited herein are hereby incorporated by reference in their entirety.

### DETAILED DESCRIPTION

is The method of this invention is accomplished by administering at least two different bifunctional agents to a subject bearing a tumor. Each of the bifunctional agents contains a binding domain that specifically binds to SSEA4 or an SSEA4 analog. The SSEA4 analog can be, e.g., SSEA3 and Globo H.

The binding domain can be an anti-SSEA4 antibody or antibody fragment. Alternatively, it can be a single chain variable domain (“scFv”) that specifically binds to SSEA4. Examples of an anti-SSEA4 antibody or antibody fragment include, but are not limited to, a fully humanized monoclonal IgG antibody and an anti-SSEA4 Fab. Indeed, exemplary anti-SSEA4 antibodies and anti-SSEA4 antibody fragments are described in US Patent Application Publication 2016/0102151.

The bifunctional agent to be administered in the method of the invention also contains an effector molecule. As mentioned above, one of the effector molecules that can be linked to the binding domain is a cytotoxic agent which interferes with cell growth and kills tumor cells typically via apoptosis. Exemplary cytotoxic agents are Diphtheria toxin, Pseudomonas exotoxin A (“PE38”), doxorubicin, methotrexate, an auristatin, a maytansine, a calicheamicin, a duocarmycin, a pyrrolobenzodiazepine dimer, and 7-ethyl-10-hydroxy-camptothecin (“SN-38”). Suitable cytotoxic agents are described in Peters et al. 2015, Biosci. Rep. 35:1-20 (“Peters et al.”); Bouchard et al. 2014, Bioorg. Med. Chem. Lett. 24:5357-5363; Panowski et al. 2014, mAbs 6:34-45; and Mazor et al. 2016, Immunol. Revs. 270:152-164.

The cytotoxic agent can be linked to the binding domain via a linker. In an embodiment, the linker is cleavable such that, upon internalization of the bifunctional agent by a tumor cell, the cytotoxic agent is cleaved from the binding domain. Examples of a cleavable linker include, but are not limited to, acid-labile small organic molecules (e.g., hydrazone), protease cleavable peptides (e.g., valine-citrulline dipeptide), and disulfide bonds. In another embodiment, the linker is not cleavable. In this case, the cytotoxic agent is released upon degradation of the binding domain linked to it. Additional examples of linkers are described in Peters et al.

If the cytotoxic agent is a protein, it can be linked to the binding domain via a peptide bond, e.g., as part of a fusion protein. In one example, PE38 is fused to the C-terminus of a V_{L} chain of an anti-SSEA4 monoclonal antibody. In another example, Diphtheria toxin is fused to the C-terminus of a V_{L} chain of an anti-SSEA4 monoclonal antibody.

As set forth above, the effector molecule contained in the bifunctional agent can be a cytokine. The cytokine, after being localized to tumor cells via the binding domain, stimulates immune cells, e.g., T cells and NK cells, to kill the tumor cells. In an embodiment, the cytokine is fused to the binding domain as part of a fusion protein. See Kiefer et al. 2016, Immunol. Revs. 270:178-192. In a different embodiment, the cytokine is linked to the binding domain via cross-links between lysine residues. Exemplary suitable cytokines include G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

The method for treating a tumor is carried out, in a particular embodiment, using a bifunctional agent containing a modified immunoglobulin Fc domain as the effector molecule. An exemplary bifunctional agent is an anti-SSEA4 monoclonal antibody having a modified Fc domain. The Fc domain can be modified such that it specifically targets the FcγRIIa receptor, the FcγRIIIa receptor, or the FcRn receptor, as compared to an unmodified Fc domain. Targeting the FcγRIIa and FcγRIIIa receptors leads to an increased cytotoxic immune response. On the other hand, targeting the FcRn receptor increases the half-life of the bifunctional agent. Modifications to the Fc domain that increase its affinity for the FcγRIIa receptor, the FcγRIIIa receptor, or the FcRn receptor are described in Moore et al. 2010, mAbs 2:181-189 and Lobner et al. 2016, Immunol. Revs. 270:113-131.

A further effector molecule that can be used in a bifunctional agent of the invention is an anti-CD3 molecule. The anti-CD3 molecule activates T cells localized to tumor cells via the binding domain of the bifunctional agent. An exemplary anti-CD3 molecule is an antibody fragment that binds specifically to CD3. In an embodiment, the anti-CD3 molecule specifically binds to CD3c. An example of a bifunctional agent having this effector molecule is an anti-SSEA4/anti-CD3 chimeric antibody. In a particular embodiment, the bifunctional agent is a scFv that specifically binds to SSEA4 fused to another scFv that specifically binds to CD3, i.e., a so-called bispecific T-cell engager.

Another effector molecule that can be used is an anti-CD16 molecule. The anti-CD16 molecule activates NK cells localized to tumor cells via the binding domain of the bifunctional agent. The anti-CD16 molecule, like the anti-CD3 molecule described in the preceding paragraph, can be an antibody fragment that binds specifically to CD16. Exemplary bifunctional agents are anti-SSEA4/anti-CD16 chimeric antibody and a scFv that specifically binds to SSEA4 fused to another scFv that specifically binds to CD16.

As disclosed, the method for treating a tumor requires administering at least two different bifunctional agents described above to a subject having a tumor. In certain embodiments, the different bifunctional agents administered have the same SSEA4 binding domain but different effector molecules. For example, a bifunctional agent that includes an anti-SSEA4 scFv linked to IL-2 is administered together with a different bifunctional agent that includes the anti-SSEA4 scFv linked to an anti-CD3 scFv.

In other embodiments, the different bifunctional agents have different SSEA4 binding domains and different effector molecules. In an example of this particular embodiment, a first bifunctional agent having an anti-SSEA4 scFv linked to IL-12 is administered together with an anti-SSEA4/anti-CD16 chimeric antibody.

Any of the anti-SSEA4 binding domains described above can be linked to any of the effector molecules also described above.

To carry out the method of this invention, i.e., treating a tumor by administering at least two different bifunctional agents, the administration routes can be oral, intravenous, injection, intrathecal, intraperitoneal, intra-arterial, and topical. Preferably, the bifunctional agents are administered intravenously or by injection.

The method for treating a tumor includes, as an optional step, determining whether cells in the tumor express SSEA4 on their surfaces. The tumor can be, but is not limited to a tumor of the breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow.

Without further elaboration, it is believed that one skilled in the art can, based on the description above, utilize the present invention to its fullest extent.

The following references, some of which have been cited above, can be used to better understand the background of the application.

Bouchard et al. 2014, Bioorg. Med. Chem. Lett. 24:5357-5363

Kiefer et al. 2016, Immunol. Revs. 270:178-192

Klinger et al. 2016, Immmunol. Revs. 270:193-208

Lobner et al. 2016, Immunol. Revs. 270:113-131

Mazor et al. 2016, Immunol. Revs. 270:152-164

Moore et al. 2010, mAbs 2:181-189

Neri et al. 2016, Curr. Opin. Immunol. 40:96-102

Panowski et al. 2014, mAbs 6:34-45

Peters et al. 2015, Biosci. Rep. 35:1-20

Vallera et al. 2013, Cancer Biotherapy and Radiopharma. 28:274-282

Woyach et al. 2014, Blood 124:3553-3560

The contents of the above references are hereby incorporated by reference in their entirety.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a tumor in a subject, the method comprising administering to the subject having a tumor at least two different bifunctional agents each of which contains a binding domain linked to an effector molecule, wherein the binding domain specifically binds to stage-specific embryonic antigen 4 (SSEA4) or an SSEA4 analog, cells in the tumor express SSEA4 or the SSEA4 analog, and the effector molecule is a cytotoxic agent, a cytokine, an immunoglobulin Fc domain, anti-CD3, or anti-CD16.

2. The method of claim 1, wherein one of the at least two different bifunctional agents contains an effector molecule selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and SN-38.

3. The method of claim 2, wherein one of the at least two different bifunctional agents contains an effector molecule selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

4. The method of claim 3, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.

5. The method of claim 2, wherein one of the at least two different bifunctional agents contains an effector molecule that is an immunoglobulin Fc domain modified to target the FcyRIIa receptor, the FcyRIIIa receptor, or the FcRn receptor.

6. The method of claim 5, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.

7. The method of claim 1, wherein one of the at least two different bifunctional agents binds specifically to SSEA4 and to CD3 or CD16.

8. The method of claim 7, wherein one of the at least two different bifunctional agents contains a single chain Fv domain (scFv) that binds specifically to SSEA4, fused to a scFv domain that binds specifically to CD3 or CD16.

9. The method of claim 8, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.

10. The method of claim 1, wherein one of the at least two different bifunctional agents contains an effector molecule selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

11. The method of claim 10, wherein one of the at least two different bifunctional agents contains an effector molecule that is an immunoglobulin Fc domain modified to target the FcyRIIa receptor, the FcyRIIIa receptor, or the FcRn receptor.

12. The method of claim 11, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.

13. The method of claim 10, wherein one of the at least two different bifunctional agents binds specifically to SSEA4 and to CD3 or CD16.

14. The method of claim 13, wherein one of the at least two different bifunctional agents contains a single chain Fv domain (scFv) that binds specifically to SSEA4, fused to a scFv domain that binds specifically to CD3 or CD16.

15. The method of claim 14, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.

16. The method of claim 15, wherein one of the at least two different bifunctional agents is a scFv that binds specifically to SSEA4 fused to a scFv domain that binds specifically to CD3 and another of the at least two different bifunctional agents contains an effector molecule that is IL-2, IL-12, IL-23, or IFNγ.

17. The method of claim 1, wherein one of the at least two different bifunctional agents contains an effector molecule that is an immunoglobulin Fc domain modified to target the FcγRIIa receptor, the FcγRIIIa receptor, or the FcRn receptor.

18. The method of claim 17, wherein one of the at least two different bifunctional agents binds specifically to SSEA4 and to CD3 or CD16.

19. The method of claim 18, wherein one of the at least two different bifunctional agents contains a single chain Fv domain (scFv) that binds specifically to SSEA4, fused to a scFv domain that binds specifically to CD3 or CD16.

20. The method of claim 19, wherein the tumor is a breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, or bone marrow tumor.
